SEQUENCE LISTING (1 sequence)

SEQ ID NO: 1 - DNA, 1216 bp, Homo sapiens
ctcattgacttccttcctgttctaactgccagtactcagaagtcagagttgagagacagaggcaccccggacagagacgtgaagcactgaataaatagatcagaatgactgaaaaagccccagagccacatgtggaggaggatgatgatgatgagctggacagcaagctcaattataagcctccaccacagaagtccctgaaagagctgcaggaaatggacaaagatgatgagagtctaattaagtacaagaaaacgctgctgggagatggtcctgtggtgacagatccgaaagcccccaatgtcgttgtcacccggctcaccctggtttgtgagagtgccccgggaccaatcaccatggaccttactggagatctggaagccctcaaaaaggaaaccattgtgttaaaggaaggttctgaatatagagtcaaaattcacttcaaagtgaacagggatattgtgtcaggcctgaaatacgttcagcacacctacaggactggggtgaaagtggataaagcaacatttatggttggcagctatggacctcggcctgaggagtatgagttcctcactccagttgaggaggctcccaagggcatgctggcgcgaggcacgtaccacaacaagtccttcttcaccgacgatgacaagcaagaccacctcagctgggagtggaacctgtcgattaagaaggagtggacagaatgaatgcatccacccctttccccacccttgccacctggaagaattctctcaggcgtgttcagcaccctgtccctcctccctgtccacagctgggtccctcttcaacactgccacatttccttattgatgcatcttttcccaccctgtcactcaacgtggtccctagaacaagaggcttaaaaccgggctttcacccaacctgctccctctgatcctccatcagggccagatcttccacgtctccatctcagtacacaatcatttaatatttccctgtcttacccctattcaagcaactagaggccagaaaatgggcaaattatcactaacaggtctttgactcaggttccagtagttcattctaatgcctagattcttttgtggttgttgctggcccaatgagtccctagtcacatcccctgccagagggagttcttcttttgtgagagacactgtaaacgacacaagagaacaagaataaaacaataactgtgtgtgttctggctgag

(19) United States
(12) Patent Application Publication
(10) Pub. No.: US 2010/0311044 A1
(43) Pub. Date: Dec. 9, 2010

(54) ASSAY KIT FOR IN-SITU HYBRIDIZATION OF RHOGDI2 GENE, METHOD THEREFOR AND USE THEREOF THE ASSAY KIT

(75) Inventors: Yunfu Zhang, Shanghai (CN); Jianying Qiu, Shanghai (CN)

Correspondence Address: AUSTIN RAPP & HARDMAN, 170 South Main Street, Suite 735, SALT LAKE CITY, UT, 84101, US

(73) Assignee: BIONATUREGEN TECHNOLOGY (SHANGHAI) CO., LTD, Shanghai (CN)

(21) Appl. No.: 12/495,565

(22) Filed: Jun. 30, 2009

(30) Foreign Application Priority Data
Jul. 22, 2008 (CN) 200810040824.6

(51) Int. Cl. C12Q 1/68

(52) U.S. Cl. 435/6

(57) ABSTRACT

An assay kit for in-situ hybridization (Rho GDP dissociation inhibitor beta, ISH) of RhoGDI2 gene, an assay method therefor and a use of the assay kit are provided. The assay kit includes: a hybridization probe, a marker, a hybridization solution, and an enhancement reagent. The hybridization probe has a sequence of SEQ ID NO. 1. The assay method includes steps of: (a) mixing the hybridization probe of the assay kit with a to-be-tested RNA on a substrate to form a hybridization complex; and (b) assaying the hybridization complex formed in the step (a). The use of the assay kit is applied the assay kit to prepare a therapeutic medicines for early metastasis of a carcinoma or a relapse disease. The assay kit of the present invention can enhance sensitivity and strengthen specificity. Meanwhile, the assay method of the present invention can increase operational convenience and simplify procedures, so that the assay method can be popularized in the medical institutions including local hospitals.

### PRIORITY CLAIM

This application claims priority to Chinese Patent Application No. 200810040824.6 filed on Jul. 22, 2008.

### BACKGROUND OF THE INVENTION

1. Field of the Invention

The present invention relates to an assay kit, more particularly to an assay kit for in-situ hybridization of RhoGDI2 (Rho GDP dissociation inhibitor beta) gene, an assay method therefor and a use of the assay kit.

2. Description of the Related Art

In China, according to related information from Chinese and international authorities, it is estimated that about 1.7 million Chinese people suffer from various carcinomas every year, about 1.6 million Chinese people die due to the carcinomas every year and total number of carcinoma patients is about 6 million. In the world, it has been estimated that about 8 million people suffer from the carcinomas every year, about 8 million people die due to the carcinomas every year and total number of carcinoma patients is about 84 million. In the year 2020, the foregoing numbers thereof are incredibly estimated to be twice.

In the year 2005, an annual report published by NIH (National Institutes of Health), NCI (National Cancer Institute), and CDC (Centers for Disease Control and Prevention) in the U.S. had concluded that mankind has lost the war of fighting against cancers, that is to say, the death rate of cancer cases is never reduced. The report also provided a plurality of possible failure factors, as follows: (1) the heterogeneous characters of tumor cells; (2) the drug-resistance of tumor cells; and (3) the incompletion for designing the anti-cancer therapies. Meanwhile, according to the report, it suggested that current diagnoses and therapies of cancers needs to be reviewed. The present invention discovers the other two important factors which suggested the blocks of reducing the death rate of cancer cases, as follows: (1) the difficulties of accurate diagnoses in the early period of cancer patients; and (2) the uncertainty of pathological mechanisms of metastasis. Traditionally medical images and other biochemical markers (e.g. protein markers) can be used to diagnose cancers, wherein a space-occupying tumor with a size smaller than 2 cm is defined as an early cancer case, while some tumors with a size smaller than 2 cm too much may have no any symptom. However, the foregoing clinical concepts have to be re-examined. In other words, the scientific definition that a tumor with a size smaller than 2 cm is diagnosed to be an early cancer case based on medical images should be inexact. From the viewpoint of cell biology, a tumor with a size about 1 cm contains 100 million tumor cells, while a tumor with a size about 2 cm may have a 3D volume containing more than 200 million tumor cells. The pathological evolution duration, which is defined from the early carcinogenetic period toward the generation of monoclonal tumor cells to the formation of tumors with a size about 2 cm, is very long, such as 1 year, 2 years, or more than 3 years. Therefore, it is very difficult to prove if the tumor is the single origin and etiological cause of carcinogenesis. Moreover, it has been clinically proven that other tumor cells can migrate to other tissue portions to clonally grow through different pathways once a tumor is formed. Once a primary origin of the tumor is cut off, recurrent tumor of other organs or multi-focal tumor thereof will be generated and migrated. Thus, it is inexact to clinically define if a tumor is in an early stage or not based on the size of the tumor when the size is small than 2 cm (In some cases, the primary origin and the metastatic origin of the tumor are found at the same time, however, such finding will not be discussed in the present invention). More importantly, when the above-mentioned diagnoses is executed, the cancer case may already be in a late stage, and this is the real reason for blocking the reduction of the death rate of cancer cases.

Nowadays, with the advance of molecular biological technologies and the development of functional genomics and carcinomatous genomics, it is feasible to carry out early cancer diagnoses with more accuracy at a genetic level. As a result, when an early metastasis or a (monoclonal) carcinogenesis occur or cancer cells initially migrate to penetrate vessel walls, an early preventive diagnosis will be available.

Dr. Dan Theodorescu, the professor of urology and molecular physiology of the University of Virginia, leads a research team to discover that RhoGDI2 (Rho GDP dissociation inhibitor beta) gene is involved in the metastasis of bladder cancer. When the RhoGDI2 gene is activated in cancer cells, the cells can generate proteins to stop the cancer cells invading other organs.

So far, high throughput gene biochips are used in the studies of RhoGDI2 gene, most of which are used for scientific researches and unsuitably applied to clinical applications. Especially, conditions of customized or individualized applications are still immature in China now.

The so-called “in-situ hybridization (ISH)” combines molecular biology with cellular chemistry to provide a technique which uses marked nucleic acids as probes to in-situ detect specific nucleic acids at tissue cells. The principle of the ISH is described as follows: under suitable conditions, allowing a hybridization between marked single-stranded nucleic acids having specific sequences and complementary single-stranded nucleic acids (e.g. target nucleic acids) in tissue cells, and then detecting the marked probes by autoradiography or immuno-cytochemistrical methods, so as to in-situ show specific DNA or RNA molecules in the tissue cells.

Generally, ISH probes use molecules having known sequences or known molecules having unknown sequences (e.g. although not all of sequences of a molecule are known, a target molecule of the molecule is definite). There are several kinds of probes that are classified into DNA probes, cDNA probes, cRNA probes, and synthesized oligo-nucleotide probes according to character differences of nucleic acids. For the convenience of tracing, the probes must be marked by suitable means for conveniently executing detections thereof. Traditional markers includes radioactive nuclides and non-radioactive markers, wherein common radioactively isotopic markers includes ³H, ³⁵S, ¹²⁵I, or ³²P. Although the radioactively isotopic markers provide advantages including higher sensitivity and clearer image background, the radioactively isotopic markers are gradually replaced by the non-isotopic markers due to harmful risks to humans and the environment. Examples of the non-isotopic markers includes biotin, digoxigenin (DIG), and fluorescein, wherein detection methods therefor are extremely sensitive

According to differences of adapted probes and targeted nucleic acids, the hybridization techniques can be classified into DNA-DNA hybridization, RNA-DNA hybridization, and RNA-RNA hybridization, each of which must be processed by five major steps: immobilization, pre-hybridization, hybridization, rinse and development.

Chinese Pat. No. 1,556,410 discloses an assay kit for fish virus and its detecting method; Chinese Pat. No. 1,680,597 discloses a fluorescent quantitative PCR assay kit of hepatitis C virus (HCV); and Chinese Pat. No. 2,918,345 disclose an oligo-nucleotide biochip for fatness related gene SNP and an assay kit therefor. However, an assay kit for in-situ hybridization of RhoGDI2 gene and related technologies therefor are never disclosed and published.

### SUMMARY OF THE INVENTION

A primary object of the present invention is to provide an assay kit for in-situ hybridization of RhoGDI2 (Rho GDP dissociation inhibitor beta) gene.

A secondary object of the present invention is to provide a method for in-situ hybridization of RhoGDI2 gene.

A third object of the present invention is to provide a use of the assay kit for in-situ hybridization of RhoGDI2 gene.

To achieve the above objects, a preferred embodiment of the present invention is carried out by technologies as follows: from inspections of large quantities of samples (such as radical adenocarcinoma in various tissues including blood cells of liver, lung, kidney, stomach, uterus, ovary, breast, colon, and etc.), it is observed that the expression of RhoGDI2 gene of patients with carcinoma metastasis generally decreases or almost zero, so that the expression of RhoGDI2 gene is an important index for clinical researches. After analyzing statistic data based on blood samples of two groups of people including 800 patients with various carcinoma (most thereof are accompanied with metastasis) for test and 800 normal individuals without cancer for comparison, the result shows that the average expression rate of RhoGDI2 gene is about 8% in carcinoma patients, while the average expression rate is 50% in the group of normal individuals, wherein the expression rates of about 600 carcinoma patients are almost zero. Meanwhile, linear relatives of two families with a high risk of heredity carcinoma are inspected, wherein the expression rates thereof are less that that of normal individuals. The RhoGDI2 gene provided by the present invention is a tumor metastasis suppressor gene which is belonged to the human homozygous gene and classified into one member of Rho gene family located at the position 12p12.3 of the human chromosome, wherein the Rho gene family is involved in functions of GTP and GDP-1 and intracellular motions. The RhoGDI2 gene is broadly expressed in tissues and organs of normal individuals, but the expression of the RhoGDI2 gene is apparently decreased or even disappeared in patients with various carcinomas and tumor metastases. The expression variation of the RhoGDI2 gene is an obvious difference for being distinguished from other tumor metastasis suppressor genes, while the RhoGDI2 gene can provide a wide-range tumor suppressor function. The assay kit of the present invention combines the technique of nucleic acid hybridization and the method of immuno-histochemistry. Meanwhile, the target of the assay kit is the RhoGDI2 gene, the synthetic probes are DNA or RNA sequences of the RhoGDI2 gene, and the inspected substrates are the RNA expression of leukocytes or tissue cells of human blood samples. The development of the in-situ hybridization (ISH) can be used to determine the level of semi-quantitative or quantitative expression of the RhoGDI2 gene. The expression of the RhoGDI2 gene can be determined according to developed colors of the immuno-histochemical development after executing the ISH. Generally, the expression of the RhoGDI2 gene of cancer patients expresses at a low level or does not express, i.e. there will be no developed color. The expression of normal individuals will reach 50%, and thus the develop color is darker. The nucleic acid hybridization according to the present invention comprises several steps of: (1) preparing samples: firstly, extracting RNA from tissue samples. The RNA samples can be directly separated by electrophoresis under a denaturation condition. Then, blotting the RNA samples, and immobilizing it by cross-linkage. (2). preparing probes: a probe is a segment of nucleic acid, which can bind to the targeted nucleic acid according to the base pairing rule. In experiments of nucleic acid hybridization, the probes must be labeled with detectable elements or molecules. Thus, through immuno-blotting the probe molecules bonded with the nucleic acid molecules on a membrane, the location of the blotted probes on the membrane (i.e. the location on an electrophoretic gel) can be showed, so as to obtain the molecular size of the nucleic acid molecules. The probe molecules used by the assay kit of the present invention is preferably digoxigenin (DIG) markers. (3). hybridizing: firstly, a pre-hybridization is executed, wherein a solution of non-specific nucleic acids is used to block non-specific binding sites on the membranes. Since the blotted nucleic acid molecules are denatured, only the marked probes need to be denatured during the hybridization. Then, the probes and the membrane are reacted under a specific temperature. Finally, rinsing out the unbound probe molecules. (4). Inspecting: a corresponding immuno-histochemical method is used to execute an inspection operation substantially comprising steps of: sample preparation, pre-hybridization, hybridization, immuno-histochemical blotting, quantitative analysis under microscope, and report of result, wherein the hybridization further comprising steps of:

(a) placing a sample into a reaction tank;

(b) providing an instrument to automatically remove liquid of the sample and automatically add a digestive solution into the sample;

(c) automatically removing liquid of the sample and automatically immobilizing the sample by the instrument;

(d) automatically removing liquid of the sample and automatically pre-hybridizing (42° C.) the sample by the instrument;

(e) automatically removing liquid of the sample and automatically rinsing the sample by the instrument;

(f) automatically removing liquid of the sample and automatically hybridizing (42° C.) the sample by the instrument;

(g) automatically removing liquid of the sample and automatically rinsing the sample by the instrument;

(h) automatically removing liquid of the sample and automatically incubating the sample with DIG-labeled antibodies (at room temperature) by the instrument;

(i) automatically removing liquid of the sample followed by automatically rinsing and developing the sample by the instrument; and

(j). taking the sample out of the instrument and mounting the sample on a slide for being analyzed be a microscope.

A plurality of embodiments according to the present invention are described more detailed as follows:

In one embodiment of the present invention, an assay kit for in-situ hybridization (ISH) of RhoGDI2 gene is provided, wherein the assay kit comprises a hybridization probe, a marker, a hybridization solution and an enhancement reagent, wherein the hybridization probe has a sequence of SEQ ID NO. 1 (Homo sapiens), as follows:

SEQUENCE LISTING

<170> PatentIn version 3.1
<210> 1
<211> 1216
<212> DNA
<213> Homo sapiens
<400> Sequence: 1

| | |
|---|---|
| ctcattgact tccttcctgt tctaactgcc agtactcaga agtcagagtt gagagacaga | 60 |
| ggcaccccgg acagagacgt gaagcactga ataaatagat cagaatgact gaaaaagccc | 120 |
| cagagccaca tgtggaggag gatgatgatg atgagctgga cagcaagctc aattataagc | 180 |
| ctccaccaca gaagtccctg aaagagctgc aggaaatgga caaagatgat gagagtctaa | 240 |
| ttaagtacaa gaaaacgctg ctgggagatg gtcctgtggt gacagatccg aaagccccca | 300 |
| atgtcgttgt cacccggctc accctggttt gtgagagtgc cccgggacca atcaccatgg | 360 |
| accttactgg agatctggaa gccctcaaaa aggaaaccat tgtgttaaag gaaggttctg | 420 |
| aatatagagt caaaattcac ttcaaagtga acagggatat tgtgtcaggc ctgaaatacg | 480 |
| ttcagcacac ctacaggact ggggtgaaag tggataaagc aacatttatg gttggcagct | 540 |
| atggacctcg gcctgaggag tatgagttcc tcactccagt tgaggaggct cccaagggca | 600 |
| tgctggcgcg aggcacgtac cacaacaagt ccttcttcac cgacgatgac aagcaagacc | 660 |
| acctcagctg ggagtggaac ctgtcgatta agaaggagtg gacagaatga atgcatccac | 720 |
| ccctttcccc acccttgcca cctggaagaa ttctctcagg cgtgttcagc accctgtccc | 780 |
| tcctccctgt ccacagctgg gtccctcttc aacactgcca catttcctta ttgatgcatc | 840 |
| ttttcccacc ctgtcactca acgtggtccc tagaacaaga ggcttaaaac cgggctttca | 900 |
| cccaacctgc tccctctgat cctccatcag ggccagatct tccacgtctc catctcagta | 960 |
| cacaatcatt taatatttcc ctgtcttacc cctattcaag caactagagg ccagaaaatg | 1020 |
| ggcaaattat cactaacagg tctttgactc aggttccagt agttcattct aatgcctaga | 1080 |
| ttcttttgtg gttgttgctg gcccaatgag tccctagtca catcccctgc cagagggagt | 1140 |
| tcttcttttg tgagagacac tgtaaacgac acaagagaac aagaataaaa caataactgt | 1200 |
| gtgtgttctg gctgag | 1216. |

In one embodiment of the present invention, the marker is selected from a radioactive nuclide or a non-radioactive marker.

In one embodiment of the present invention, the radioactive nuclide is selected from ³H, ³⁵S, ¹²⁵I, or ³²P.

In one embodiment of the present invention, the non-radioactive marker is selected from biotin, digoxigenin (DIG), alkaline phosphatase, horseradish peroxidase (HRP), or fluorescein.

In one embodiment of the present invention, the non-radioactive marker is preferably selected from digoxigenin.

In one embodiment of the present invention, the enhancement reagent in the hybridization solution is selected from antibodies of alkaline phosphatase;

In one embodiment of the present invention, an assay method for in-situ hybridization of RhoGDI2 gene is provided, wherein the method comprises steps of:

(a) mixing the hybridization probe of the foregoing assay kit with a to-be-tested RNA on a substrate to form a hybridization complex; and

(b) assaying the hybridization complex formed in the step (a);

wherein conditions for forming the hybridization complex in the step (a) comprises: the temperature for nucleic acid hybridization is 42° C.; and the duration for the nucleic acid hybridization is 16-24 hours; and the substrate is selected from a blood monocyte sample.

In one embodiment of the present invention, a use of an assay kit for in-situ hybridization of RhoGDI2 gene is provided, wherein the assay kit is applied to prepare a therapeutic medicines for early metastasis of a carcinoma or a relapse disease.

In one embodiment of the present invention, the carcinoma is selected from liver cancer, lung cancer, stomach cancer, breast cancer, colon cancer, prostate cancer, uterus cancer, or pancreatic cancer.

According to the present invention, the assay kit for in-situ hybridization of RhoGDI2 gene, the assay method therefore and the use of the assay kit can provide several advantages, as follows:

The assay kit provided by the present invention can enhance sensitivity and strengthen specificity. Meanwhile, the assay method of the present invention can increase operational convenience and simplify procedures, so that the assay method can be popularized in the medical institutions including local hospitals. In comparison with traditional assay method of RhoGDI2 gene, which generally use high throughput gene biochips and are only applied to for scientific researches without clinical application, the assay method of the present invention can provide a clinical improvement to trace and inspect the carcinogenesis and the carcinoma metastasis in the pathological evolution duration as early as possible. The assay kit of the present invention is apparently different from other markers for inspecting carcinoembryonic proteins and inspection means of medical images. The present invention can observe and measure the abnormal expression of RhoGDI2 gene at genetic level. Before the inspection means of medical images finds the relapse symptom of a space-occupying tumor or any abnormalities of biochemical indices of tumors become observable (i.e. prior to the formation of tumors), the abnormal messages of genetic expression could be collected according to the present invention, and thus the early diagnosis and early forecast of metastasis relapse after treatment for carcinoma patients can be made. As a result, it is possible to carry out earlier diagnosis, prophylaxis and therapies, while it is feasible to early find the source of the carcinoma for curing completely the carcinomas.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is an image showing results of in-situ hybridization (ISH) of RhoGDI2 gene based on blood samples from normal individuals;

FIG. 2 is an image showing results of in-situ hybridization (ISH) of RhoGDI2 gene based on blood samples from carcinoma patients;

FIG. 3 is a statistic block diagram showing expressions of RhoGDI2 gene of all ages in normal individuals;

FIG. 4 is a statistic curved graph showing expressions of RhoGDI2 gene of all ages in normal individuals, similar to FIG. 3;

FIG. 5 is a statistic block diagram showing expressions of RhoGDI2 gene of all ages in various carcinoma patients; and

FIG. 6 is a statistic curved graph showing expressions of RhoGDI2 gene of all ages in various carcinoma patients.

### DESCRIPTION OF THE PREFERRED EMBODIMENTS

The present invention will be better understood by reference to the following examples, which are provided as exemplary of the invention, and not by way of limitation, and any changes and modifications to the described embodiment can be carried out without departing from the scope and the spirit of the invention.

### Example 1

### An Assay Kit for In-Situ Hybridization (ISH) of RhoGDI2 Gene

An assay kit for in-situ hybridization (ISH) of RhoGDI2 gene comprises a hybridization probe, a marker, a hybridization solution, an enhancement reagent; wherein the hybridization probe has a sequence of SEQ ID NO. 1, as shown hereinafter. The hybridization probe is marked by the marker selected from digoxigenin (DIG). Other solutions and samples of the assay kit are listed as follows:

| | | | | |
|---|---|---|---|---|
| Digestive solution | 100 | μl/tube | 1 tube/kit | Colorless transparent liquid |
| Reserving solution | 100 | μl/tube | 1 tube/kit | Colorless transparent liquid |
| Pre-hybridization | 1300 | μl/tube | 2 tube/kit | Colorless transparent liquid |
| solution | | | | |
| Hybridization solution | 10 | μl/tube | 1 tube/kit | Colorless transparent liquid |
| with sense probe | | | | |
| Hybridization solution | 10 | μl/tube | 1 tube/kit | Colorless transparent liquid |
| with anti-sense probe | | | | |
| Blocking solution | 1000 | μl/tube | 1 tube/kit | Colorless transparent liquid |
| Antibodies of alkaline | 1 | μl/tube | 1 tube/kit | Colorless transparent liquid |
| phosphatase | | | | |
| Developing reagent A | 175 | μl/tube | 1 tube/kit | Yellow liquid |
| Developing reagent B | 320 | μl/tube | 1 tube/kit | Colorless transparent liquid |
| 10x Buffer solution I | 90 | ml/tube | 1 tube/kit | Light yellow or Colorless |
| | | | | transparent liquid |
| 10x Buffer solution II | 80 | ml/tube | 1 tube/kit | Light yellow or Colorless |
| | | | | transparent liquid |
| 10x Buffer solution III | 20 | ml/tube | 3 tube/kit | Light yellow or Colorless |
| | | | | transparent liquid |
| 10x Buffer solution IV | 90 | ml/tube | 1 tube/kit | Light yellow or Colorless |
| | | | | transparent liquid |
| Immobilizing solution | 90 | ml/tube | 1 tube/kit | Colorless transparent liquid |
| Samples (for positive | | | 6 slides/kit | |
| control) | | | | |

Detailed description of the foregoing reagents: (all chemicals are purchased from SIGMA)

(1) Digestive Solution:

20 mg/ml protease K prepared by 100 mg protease K added into 5 ml DEPC-H20

(2) Reserving Solution:

0.2 g glycine added into 1 ml 1× buffer solution I

(3) Pre-Hybridization Solution:

7.5 ml 1× buffer solution II;

3 ml 50× Digestive solution;

750 μl yeast t-RNA (10 mg/ml);

682 μl SALMON TESTES DNA (11 mg/ml);

3 ml EDTA (0.04 M); and

15 ml formamide (50%)

(4) Blocking Solution:

0.03 g blocking (Rosch) added into 1 ml 1× buffer solution III

(5) 10× Buffer Solution I: (PH 7.1-7.4)

80 g NaCl;

360 g Na2HPO4.12H20;

2 g KCl; and

2 g KH2PO4

Adding triple-distilled water up to 1 L, and being autoclaved.

(6) 10× Buffer Solution II: (PH 7.0)

175.3 g NaCl;

88.2 g Sodium citrate; and

several drops HCl

Adding triple-distilled water up to 1 L, and being autoclaved.

(7) Buffer III: (PH 7.9)

121.1 g Tris;

87.66 g NaCl; and

about 60 ml HCl

Adding triple-distilled water up to 1 L, and being autoclaved.

(8) Buffer Solution IV:

(a) 1 M Tris-HC1 (PH 9.5):

121.1 g Tris added into 3 ml HC1;

adding 900 ml water;

adjusting the PH value to 9.5;

adding water up to 1 L, and being autoclaved

(b) 1 M NaCl:

58.44 g NaCl,

adding water up to 1 L, and being autoclaved

(c) 0.5 M MgC12:

101.65 g MgC12.6H2O

adding water up to 1 L, and being autoclaved

(9) Immobilizing Solution:

40 g paraform-aldehyde

adding 1× buffer solution I up to 1 L;

slightly heating (about 50-60); and

stiring to be completely dissolved.

(10) Developing Reagent A:

1 g NBT added into 11.44 ml DMF(70%).

(11) Developing Reagent B:

1 g NBT added into 30 ml DMF(100%).
The assay kit of the present invention can be used for several individuals or for only one individual.

### Example 2

### An Assay Method For In-Situ Hybridization (ISH) of RhoGDI2 gene

I. Preparation of Specimens:

(1) Adding 4.5 ml separation solution of lymph cells into a 10 ml centrifugal tube, and then slowly dropping 3 ml anticoagulated blood into the centrifugal tube having the separation solution of lymph cells (anticoagulated blood: separation solution =1:1.5). After this, centrifuging the centrifugal tube about 10 min (2000 rpm/min).

(2) Drawing out a leukocyte solution in an intermediate layer in the centrifugal tube, and loading the leukocyte solution into another centrifugal tube. Then, introducing 1× buffer solution I (with about 2-folded volume of the leukocyte solution) into the centrifugal tube, and mixing uniformly. After this, centrifuging the centrifugal tube about 10 min (1500 rpm/min).

(3) Removing an upper clear suspension in the centrifugal tube to remain precipitate, introducing 1× buffer solution I (with about 2-folded volume of the precipitate) into the centrifugal tube, and mixing uniformly. After this, centrifuging the centrifugal tube about 10 min (1500 rpm/min).

(4) Removing an upper clear suspension in the centrifugal tube to remain the precipitate, and wiping out the residual solution on an opening of the centrifugal tube with tissue. Then, re-suspending the precipitae to become a suspension which will be dropped upon a glass slide and dried spontaneously. (the slide could be prepared by an automatic slide maker in some hospitals). Generally, 3 ml blood can be used to prepare 4 slides.

(5) preparing 40 ml immobilizing solution (4%) to immobilize the slide in a glass container about 30 min, and then rinsing the slide by 1× buffer solution I about 5 min, wherein each of the glass container can receive 16 slides.

(6) keeping the slides at −20° C. for waiting the following experiments.

II. Preparation of Operational Solution by Using Reagents in the Assay Kit of Embodiment 1:

(1) Diluting 10× buffer solution I with 10 times triple-distilled water into 1× buffer solution I.

(2) Diluting 20× buffer solution II with 10 times triple-distilled water into 2× buffer solution II; diluting 20× buffer solution II with 100 times triple-distilled water into 0.2× buffer solution II; and diluting 20× buffer solution II with 200 times triple-distilled water into 0.1× buffer II.

(3) Diluting 10× buffer solution III with 10 times triple-distilled water into 1× buffer solution III.

(4) Diluting 10× buffer solution IV with 10 times triple-distilled water into 1× buffer solution IV (that is, mixing 10 ml 1× buffer solution I, 10 ml 1× buffer solution II, 10 ml 1× buffer solution III, and adding triple-distilled water up to 100 ml for being 1× buffer solution IV).

III. Experiment:

(1) Preparing two slides of each patient (further preparing the other two slides for re-check) and two slides of positive control (a pair of slides of positive control for each one of tests).

(2) Loading 20 ml diluted digestive solution into a glass container (100 μl digestive solution added with 199.9 ml 1× buffer solution I to be the diluted digestive solution). Preparing a water bath (37° C.) for preheating the glass container about 10 min. Then, placing 16 slides in the glass container and heating at 37° C. about 12 min. After this, rinsing the slides by 1× buffer solution I about 5 min.

(3) Rinsing the slides by 0.2% diluted reserving solution (1 ml reserving solution added with 199 ml 1× buffer solution Ito be the diluted reserving solution) about 10 min. Rinsing the slides by triple-distilled water about 5 min, wherein all of the above steps are executed in the glass container. After this, drying the slides spontaneously.

(4) Placing the slides into a humidity-keeping box followed by adding pre-hybridization solution (20 μl per slide). Then, covering the slides by cover slips, and sealing the humidity-keeping box. After this, placing the humidity-keeping box into 42° C. water bath more than 3 hours.

(5) Taking the slides out of the glass container, and removing the cover slips from the slides. Then, placing the slides into another glass container, and rinsing the slides by 70%, 90%, 95% ethanol about 2 min sequentially. After this, drying the slides spontaneously.

(6) Placing the slides into another humidity-keeping box, wherein two slides from each patient, one is dropped with hybridization solution of sense probe (20 μl/slide), and the other is dropped with hybridization solution of antisense probe (20 μl/slide). Then, covering the slides with cover slips, and sealing the humidity-keeping box. After this, placing the humidity-keeping box into 42° C. water bath about 16-24 hours.

(7) Taking the slides out of the humidity-keeping box, removing the cover slips, and placing the slides into another glass container. Rinsing the slides by 2× buffer solution II twice in 42° C. water bath about 15 min each time. Rinsing the slides by 0.2× buffer solution II twice in 42° C. water bath about 15 min each time. Rinsing the slides with 0.1× buffer solution II twice in 42° C. water bath about 15 min each time.

(8) Rinsing the slides with 1× buffer solution III about 30 sec, and taking the slides out of the glass container. After this, drying the slides spontaneously.

(9) Placing the slides into another humidity-keeping box, and adding 0.5% diluted blocking solution (1 ml blocking solution added with 5 ml 1× buffer solution III)(100 μl/slide). Then, sealing the humidity-keeping box, and reacting at room temperature about 30 min.

(10) Taking the slides out of the humidity-keeping box, and rinsing the slides by 1× buffer solution III about 30 sec. After this, drying the slides spontaneously.

(11) Placing the slides into another humidity-keeping box, and adding antibodies of alkaline phosphatase (diluted by 1.8 ml 1× buffer solution III)(100 μl/slide) into the humidity-keeping box. Then, sealing the humidity-keeping box, and reacting at room temperature about 30 min.

(12) Taking the slides out of the humidity-keeping box, and rinsing the slides by 1× buffer solution III 3 times (15 min each time). After this, drying the slides spontaneously.

(13) Rinsing the slides by 1× buffer solution IV about 2 min, and then adding the developing reagent on the slides (73.3 μl developing reagent A and 157.5 μl developing reagent B are uniformly mixed with 30 ml 1× buffer solution IV) at room temperature in dark more than 12 hours.

(14) Rinsing the slides by triple-distilled water about 5 min, and drying the slides spontaneously. Covering the slides by cover slips and observing the slides under oil immersion (uniform mixture of glycerol and 10% 1× buffer solution I) by oil lenses of a microscope.

IV. Results and Discussion:

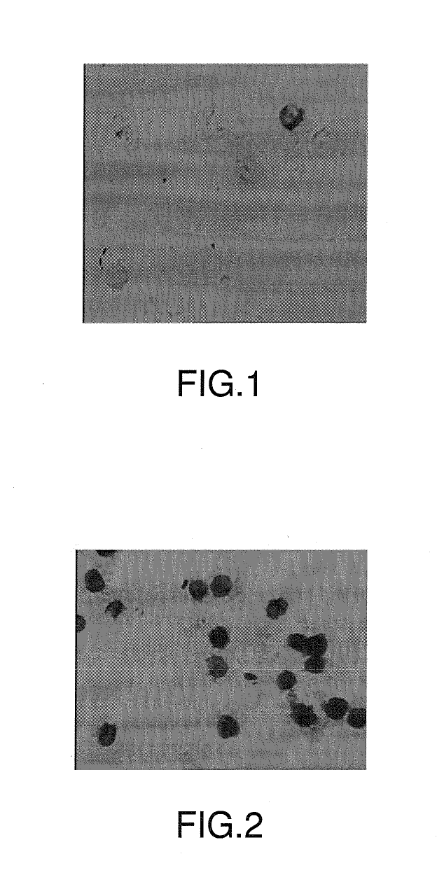

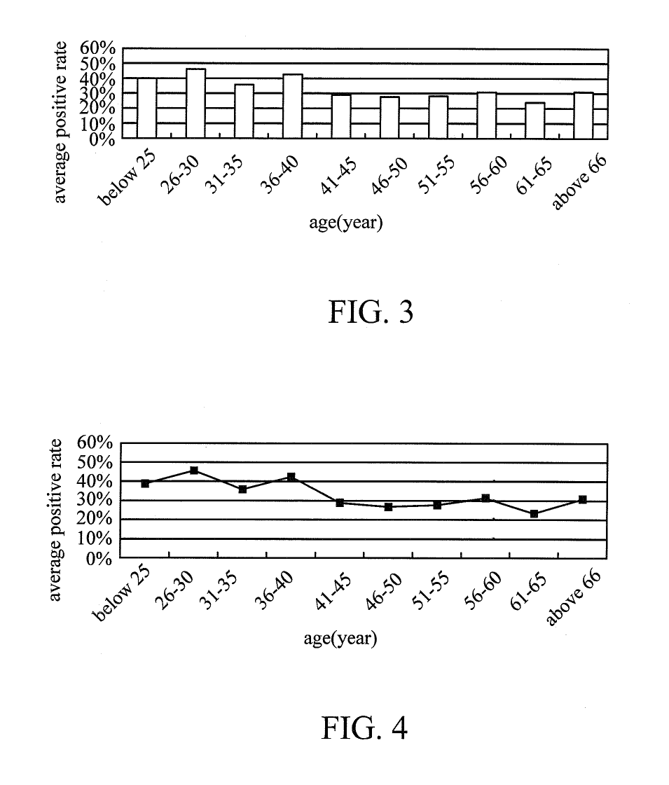

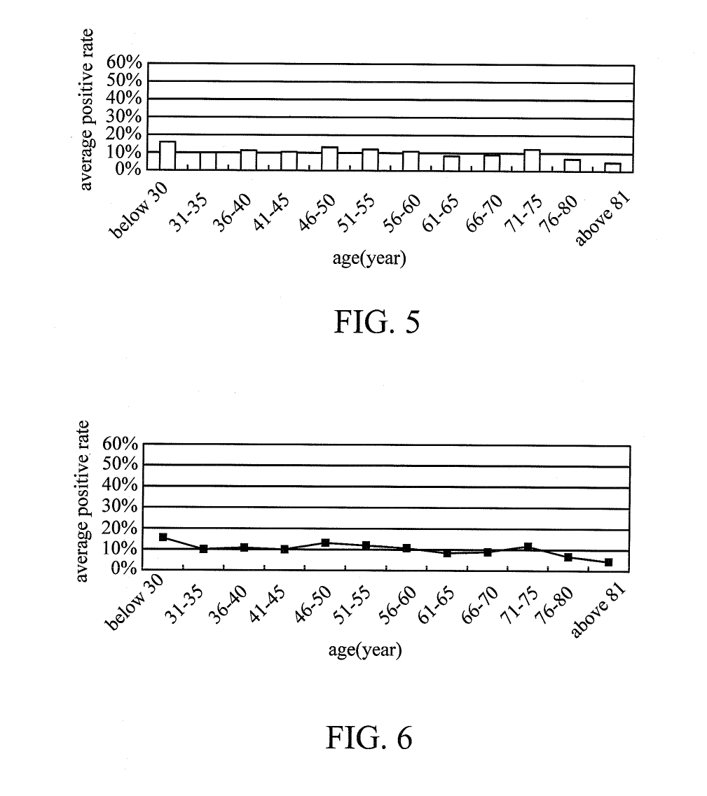

The percentage of purple-stained cells among each 100-300 cells is counted under the microscope. The slides of positive control processed by the hybridization solution with antisense probe should have more than 80% purple-stained cells. All of the re-check slides of negative internal controls processed by the hybridization solution with sense probe should only have colorless cells. The DIG-marked cDNA, RNA, and oligo-nucleotide probes not only have advantages of biotin-marked probes, but also can overcome disadvantages caused by the interruptions of endogenous biotin in tissues during the process of ISH based on biotin-marked probes. The hybridization probe is hybridized with to-be-tested RNA samples from leukocytes of human blood, and then develops the slides by immuno-histochemical methods. After this, the slides can be observed under the microscope, and then the existence and location of mRNA will be showed. Thus, the quantity of expression of target gene can be decided by the number of the stained cells. The assay method of the present invention is an ISH technology of nucleic acid for measuring the expression quantity of RhoGDI2 gene in substrate cells, in order to determine if the carcinogenesis or carcinoma metastasis occurs. The clinical researches show that RhoGDI2 gene has a wide-range tumor suppressor function. Referring to FIG. 1, an image showing results of in-situ hybridization (ISH) of RhoGDI2 gene based on blood samples from normal individuals is illustrated. Referring to FIGS. 3 and 4, a statistic block diagram and a statistic curved graph showing expressions of RhoGDI2 gene of all ages in normal individuals are illustrated, respectively. As shown, the expression of RhoGDI2 gene in normal individuals is relatively high. Referring to FIG. 2, an image showing results of in-situ hybridization (ISH) of RhoGDI2 gene based on blood samples from carcinoma patients is illustrated. Referring to FIGS. 5 and 6, a statistic block diagram and a statistic curved graph showing expressions of RhoGDI2 gene of all ages in carcinoma patients are illustrated, respectively. As shown, if the expression of RhoGDI2 gene is relatively low or even zero, it represents that the carcinoma may relapse, metastasize or expand, so that diagnosis messages can be obtained. When the detected expression of RhoGDI2 gene is lower than a normal control value, it is possible to presume if the individual is in an early metastasis stage or has a risk of metastasis in advance, wherein the foregoing carcinoma comprises: lung cancer, stomach cancer, breast cancer, colon cancer, prostate cancer, uterus cancer, and pancreatic cancer.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. An assay kit for in-situ hybridization of RhoGDI2 (Rho GDP dissociation inhibitor beta) gene, comprising:
a hybridization probe;
a marker;
a hybridization solution; and
an enhancement reagent;
where the hybridization probes has a sequence of SEQ ID NO. 1.

2. The assay kit of claim 1, wherein the marker is selected from a radioactive nuclide or a non-radioactive marker.

3. The assay kit of claim 2, wherein the radioactive nuclide is selected from ³H, ³⁵S, ¹²⁵I, or ³²P.

4. The assay kit of claim 2, wherein the non-radioactive marker is selected from biotin, digoxigenin (DIG), alkaline phosphatase, horse-radish peroxidase (HRP), or fluorescein.

5. The assay kit of claim 4, wherein the non-radioactive marker is digoxigenin.

6. The assay kit of claim 1, wherein the enhancement reagent in the hybridization solution is selected from antibodies of alkaline phosphatase.

7. An assay method for in-situ hybridization of RhoGDI2 gene, comprising steps of:
(a) mixing the hybridization probe of the assay kit of claim 1 with a to-be-tested RNA on a substrate to form a hybridization complex; and
(b) assaying the hybridization complex formed in the step (a).

8. The assay method of claim 7, wherein conditions for forming the hybridization complex in the step (a) comprises:
the temperature for nucleic acid hybridization being 42° C.;
the duration for nucleic acid hybridization being 16-24 hours; and
the substrate selected from a blood monocyte sample.

9. A use of the assay kit for in-situ hybridization of RhoGDI2 gene of claims 1, wherein the assay kit is applied to prepare a therapeutic medicines for early metastasis of a carcinoma or a relapse disease.

10. The use of claim 9, wherein the carcinoma is selected from liver cancer, lung cancer, stomach cancer, breast cancer, colon cancer, prostate cancer, uterus cancer, or pancreatic cancer.

11. A use of the assay kit for in-situ hybridization of RhoGDI2 gene of claims 7, wherein the assay kit is applied to prepare a therapeutic medicines for early metastasis of a carcinoma or a relapse disease.

12. The use of claim 11, wherein the carcinoma is selected from liver cancer, lung cancer, stomach cancer, breast cancer, colon cancer, prostate cancer, uterus cancer, or pancreatic cancer.
